(19) United States
(12) Patent Application Publication
(10) Pub. No.: US 2009/0143807 A1
(43) Pub. Date: Jun. 4, 2009

(54) Percutaneous Devices for Separating Tissue, Kits and Methods of Using the Same

(75) Inventor: Paul Sand, San Carlos, CA (US)

Correspondence Address: WILSON SONSINI GOODRICH & ROSATI, 650 PAGE MILL ROAD, PALO ALTO, CA, 94304-1050, US

(73) Assignee: Vertos Medical, Inc., a Delaware Corporation, San Jose, CA (US)

(21) Appl. No.: 12/276,326

(22) Filed: Nov. 22, 2008

Related U.S. Application Data
(60) Provisional application No. 60/991,946, filed on Dec. 3, 2007.

(51) Int. Cl. A61B 17/32; A61B 19/00

(52) U.S. Cl. 606/170; 606/167; 128/898

(57) ABSTRACT

The disclosure describes devices for separating a first tissue from a second tissue at a surgical site, comprising, a hollow body having a distal end and a proximal end, wherein the distal end further comprises an upper separation member extendable laterally from hollow body at a first angle; an inner member slidably disposed within the hollow body, wherein the distal end of the inner member has a lower separation member extendable laterally from inner member at a second angle, wherein the upper separation member and the lower separation member have a first configuration and a second configuration into a tissue in the working zone on the first lateral side of the median plane. Additionally, methods for using the device and kits containing the device are disclosed.

### CROSS-REFERENCE

This application claims the benefit of U.S. Provisional Application No. 60/991,946 filed Dec. 3, 2007, which application is incorporated herein by reference.

### BACKGROUND

1. Field of the Invention

The present invention relates generally to minimally invasive devices, systems methods and kits for treating spinal disorders; including devices, systems, methods and kits that use imaging guidance. More particularly, the present invention relates to devices, systems, methods and kits to reduce stenosis and increase the cross-sectional area of the spinal canal available for the spinal cord. Still more particularly, the present invention relates to devices, systems, methods and kits to percutaneously separate tissue such as portions of an enlarged ligamentum flavum from the lamina of a spine.

2. Background of the Invention

Back pain is a common ailment. In many cases, the pain severely limits a person's functional ability and quality of life. Back pain interferes with work, routine daily activities, and recreation. It is estimated that Americans spend $50 billion each year on low back pain alone. It is the most common cause of job-related disability and a leading contributor to missed work. Spinal stenosis, a condition that results from narrowing of the spinal canal causing nerve pinching, leads to persistent pain in the buttocks, limping, lack of feeling in the lower extremities, and decreased physical activity. Spinal stenosis is considered a silent epidemic and occurs with an incidence of between 4% and 6% (or more) of adults aged 50 and older. It is also the most frequent reason cited for back surgery in patients aged 60 and older. Currently, it is estimated that as many as 400,000 Americans, most over the age of 60, may already be suffering from the symptoms of lumbar spinal stenosis according to The American Association of Neurological Surgeons (AANS) and The Congress of Neurological Surgeons (CNS). This number is expected to grow as members of the baby boom generation begin to reach their 60s over the next decade. Moreover, according to the U.S. Census Bureau, people over 60 will account for 18.7% of the domestic population in 2010 versus 16.6% in 1999.

Lumbar spinal stenosis is often defined as a dural sac cross-sectional area less than 100 mm² or an anterior-posterior (AP) dimension of the canal of less than 10-12 mm for an average male. The source of many cases of lumbar spinal stenosis is thickening of the ligamentum flavum. Spinal stenosis may also be caused by subluxation, facet joint hypertrophy, osteophyte formation, underdevelopment of spinal canal, spondylosis deformans, degenerative intervertebral discs, degenerative spondylolisthesis, degenerative arthritis, ossification of the vertebral accessory ligaments and the like. A less common cause of spinal stenosis, which usually affects patients with morbid obesity or patients on oral corticosteroids, is excess fat in the epidural space. The excessive epidural fat compresses the dural sac, nerve roots and blood vessels contained therein and resulting in back, leg pain and weakness and numbness of the legs. Spinal stenosis may also affect the cervical and, less commonly, the thoracic spine.

Patients suffering from spinal stenosis are typically first treated with a conservative approach. A more conservative approach is a combination of rest, support devices, physical therapy, and pain medications - including anti-inflammatory medications and epidural steroid injections. This treatment is normally given over the initial months after diagnosis in hope that it will correct the problem without requiring more drastic measures. When the pain/discomfort continues, a surgical procedure is discussed and pursued if the patient and their physician think it will improve the patient's quality of life. These conservative treatment options frequently fail. If symptoms are severe, surgery is required to decompress the spinal cord and nerve roots. Surgical options are invasive and include decompression or laminectomy, laminotomy, foramitomony and spinal fusion.

In some conventional surgical approaches to correct stenosis in the lumbar region, an incision is made in the back and the muscles and supporting structures are stripped away from the spine, exposing the posterior aspect of the vertebral column. The thickened ligamentum flavum is then exposed by removal of a portion of the vertebral arch, often at the laminae, covering the back of the spinal canal (laminectomy). The thickened ligamentum flavum ligament can then be excised by sharp dissection with a scalpel or punching instruments such as a Kerrison style punch that is used to remove small chips of tissue. The procedure is performed under general anesthesia. Patients are usually admitted to the hospital for approximately five to seven days depending on the age and overall condition of the patient. Patients usually require between six weeks and three months to recover from the procedure. Further, many patients need extended therapy at a rehabilitation facility to regain enough mobility to live independently. The risks associated with surgery include bleeding, blood clots and dural tears. In some cases surgical intervention fails to relieve symptoms, or the symptoms return over time.

Much of the pain and disability after an open laminectomy results from the tearing and cutting of the back muscles, blood vessels, supporting ligaments, and nerves that occurs during the exposure of the spinal column. Also, because the spine stabilizing back muscles and ligaments are stripped and detached from the spine during the laminectomy, these patients frequently develop spinal instability post-operatively.

Minimally invasive techniques offer an important potential for less post-operative pain and faster recovery compared to traditional open surgery. Percutaneous interventional spinal procedures can be performed with local anesthesia, thereby sparing the patient the risks and recovery time required with general anesthesia. In addition, there is less damage to the paraspinal muscles and ligaments with minimally invasive techniques, thereby reducing pain and preserving these important stabilizing structures.

Various techniques for minimally invasive treatment of the spine are known. Microdiscectomy is performed by making a small incision in the skin and deep tissues to create a portal to the spine. A microscope is then used to aid in the dissection of the adjacent structures prior to discectomy. The recovery for this procedure is much shorter than traditional open discectomies. Percutaneous discectomy devices with fluoroscopic guidance have been used successfully to treat disorders of the disc but not to treat spinal stenosis or the ligamentum flavum directly. Arthroscopy or direct visualization of the spinal structures using a catheter or optical system have also been proposed to treat disorders of the spine including spinal stenosis, however these devices still use miniaturized standard surgical instruments and direct visualization of the spine similar to open surgical procedures. These devices and techniques are limited by the small size of the canal and these operations are difficult to perform and master. In addition, these procedures are painful and often require general anesthesia. Further, the arthroscopy procedures are time consuming and the fiber optic systems are expensive to purchase and maintain.

Still further, because the nerves of the spinal cord pass through the spinal canal directly adjacent to and anterior to the ligamentum flavum, any surgery, regardless of whether open or percutaneous, includes a risk of damage to the nerves of the spinal cord.

Hence, it remains desirable to provide simple methods, techniques, and devices for treating spinal stenosis and other spinal disorders without requiring open surgery. It is further desired to provide a system whereby the risk of damage to the dural sac containing the spinal nerves may be reduced.

### SUMMARY OF THE INVENTION

Methods, devices and kits for separating ligament are described herein. Other aspects and features of the devices and methods will be described in more detail below. As will be appreciated by those skilled in the art, the devices and methods can be used in connection with a wide variety of tissue. However, for purposes of illustration, and without limitation, the devices and methods are described in the context of use within the spine.

An aspect of the invention is directed to devices for separating a first tissue from a second tissue at a surgical site. Suitable devices comprise: a hollow body having a distal end and a proximal end, wherein the distal end further comprises an upper separation member extendable laterally from the hollow body at a first angle; an inner member having a distal end and a proximal end and slidably disposed within the hollow body, wherein the distal end of the inner member has a lower separation member extendable laterally from the inner member at a second angle and wherein the upper separation member and the lower separation member have a first configuration and a second configuration. In some aspects, the hollow body can be adapted and configured to provide for a separation means that is laterally extendable from the hollow body. The devices can be configured such that the first angle is greater than 90 degrees. Additionally, the second angle can, in some cases, be the same as the first angle. In any configuration of these devices, the hollow body and the inner member can be configured such that they are substantially tubular. Moreover, the devices can be configured such that the upper separation member further comprises an outer face and an inner face and the lower separation member further comprises an outer face and an inner face and further wherein the separation members are constructed and arranged so that the inner face of the lower separation member is spaced between 3 and 16 mm from the inner face of the outer separation member in the first configuration. Separation members can be any suitable means for separating. Moreover, devices can also be configured to include a handle attachable to the proximal end of the hollow body. Furthermore, devices can be configured such that the upper separation member and the lower separation member have distal ends which are beveled. The upper separation member and the lower separation member can also be configured in any of the devices such that the members are angled to form a sharpened tip. In some aspects means for moving the upper separation member and the lower separation member between the open configuration and the closed configuration, the means being located at the proximal ends of the hollow body and the inner member. In still other aspects, an actuator can be provided that is coupled to the inner member. The actuator can, for example, be a motor or any other suitable mechanism or means for actuating. In some configurations of the devices, the motor is disposed within the handle. The devices disclosed herein are configured for use with connective tissue. In some aspects, the the first tissue is ligament and the second tissue is bone; in other aspects, the first tissue is a laminae of a vertebral body and the second tissue is a ligamentum flavum.

Another aspect of the invention is directed to a kit for tissue separation. Suitable kits comprise, for example, the devices disclosed herein and and packaging. Additional components of suitable kits include, but are not limited to, for example, one or more injectable media, such as a contrast medium; one or more hydrophillic-lipophillic block copolymer gels; one or more guides adaptable for use with the device.

Yet another aspect of the invention is directed to methods for treating stenosis in a spine of a patient with reference to a median plane of the patient. The methods comprise, for example, the steps of: compressing a dural sac in the region of interest by injecting a fluid to form a safety zone and establish a working zone in the region of interest, the safety zone lying between the working zone and the dural sac; percutaneously accessing an epidural space in the region of interest on a first lateral side of a median plane; and inserting a device for separating a first tissue from a second tissue at a surgical site, comprising, a hollow body having a distal end and a proximal end, wherein the distal end further comprises an upper separation member extendable laterally from hollow body at a first angle; an inner member having a distal end and a proximal end and slidably disposed within the hollow body, wherein the distal end of the inner member has a lower separation member extendable laterally from inner member at a second angle, wherein the upper separation member and the lower separation member have a first configuration and a second configuration into a tissue in the working zone on the first lateral side of the median plane. The methods can also include, where appropriate or clinically useful, the step of generating at least one view of a portion of a spinal canal in a region of interest. Where a view is obtained, at least one view can be used to position the tissue excision device during at least part of the step of inserting. In at least some methods, at least a portion of a patient's ligamentum flavum occupies the working zone in the region of interest. Moreover, the step of using the tissue separation device to percutaneously reduce a stenosis can be performed on the first lateral side of the median plane. Some steps can include using the at least one view to position the tissue excision device during at least part of the step of using the tissue excision device. In some aspects the steps can include removing at least a portion of the ligamentum flavum in the region of interest. Still other methods can include the step of using the tissue excision device to percutaneously reduce a stenosis on a second lateral side of the median plane different than the first lateral side. Furthermore, the steps can comprise using the at least one view to position the tissue excision device during at least part of the step of using the tissue excision device.

Another aspect of the invention is directed to the use of any of the devices disclosed herein to separate tissue and/or reducing stenosis in the spinal canal.

Embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a lateral elevation view of a normal human spinal column;

FIG. 2A is a superior view of a normal human lumbar vertebra; FIG. 2B is a cross-section of the spine within the spinal canal with the dural sac and a normal (un-stenosed) ligamentum flavum therein;

FIG. 3 is a lateral elevational view of two vertebral bodies forming a functional spinal unit;

FIG. 4 is a posterolateral oblique view of a vertebrae from a human spinal column;

FIG. 5 is a perspective view of the anatomical planes of the human body;

FIGS. 6A-C are side views of tissue separation devices including the distal end;

FIG. 7 illustrates a tissue separation device of FIG. 6A with the operation features located within the device;

FIGS. 8A-B are cross-sectional views the distal end of an embodiment of a tissue separation device in a closed position and in an opened position;

FIGS. 9A and 9B are side views of alternative embodiments for the separation members of FIGS. 8A and 8B;

FIGS. 10A and 10B are top views of alternative embodiments for the separation members of FIGS. 8A and 8B;

FIG. 11A is an enlarged cross-section of a vertebral foramen, showing a safety zone created by compression of the dural sac; FIG. 11B is the cross-section of FIG. 11A, showing a tissue separation tool positioned in the ligamentum flavum using an ipsilateral approach; FIG. 11C is the cross-section of FIG. 11A, showing a tissue separation tool positioned in the ligamentum flavum using a minimally invasive decompression procedure;

FIG. 12A is a partial cross-section of the lumbar portion of the vertebral column; FIGS. 12B-D are the cross-sections of FIG. 12A, showing the orientation of a tool relative to the vertebral column; FIG. 12E is the cross-section of FIG. 12A, showing the orientation of an instrument relative to the vertebral column; and

FIGS. 13A and 13B are side schematic views illustrating the device of FIG. 6 separating a ligamentum flavum from a vertebral lamina.

### DETAILED DESCRIPTION OF THE PREFERRED EMBODIMENTS

The invention relates generally to devices, apparatus or mechanisms that are suitable for use within a human body to restore, modify and/or augment tissue, and systems therefor. As will be appreciated by those skilled in the art, tissue is an aggregation of morphologically similar cells and associated intercellular matter acting together to perform one or more specific functions in the body. There are four basic types of tissue: muscle, nerve, epidermal, and connective (which includes bone and ligament).

For purposes of illustrating the usefulness of the invention, the invention is described in the context of treating spinal pathologies. However, persons of skill in the art will appreciate that the devices can be used in conjunction with other pathologies without departing from the scope of the invention. In some instances the devices can include devices designed to separate body parts or structure. The devices, apparatus or mechanisms are configured such that the devices can be formed from parts, elements or components which alone or in combination comprise the device. The devices can also be configured such that one or more elements or components are formed integrally to achieve a desired physiological, operational or functional result such that the components complete the device. Functional results can include the surgical restoration and functional power of a patient, controlling, limiting or altering the functional power of a patient, and/or eliminating the functional power of a patient by preventing joint motion. Portions of the device can be configured to replace or augment existing anatomy and/or implanted devices, and/or be used in combination with resection or removal of existing anatomical structure.

### I. Anatomical Review

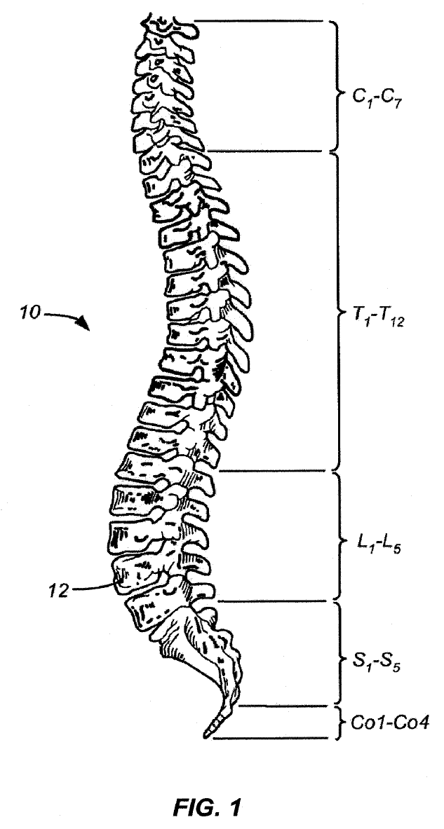

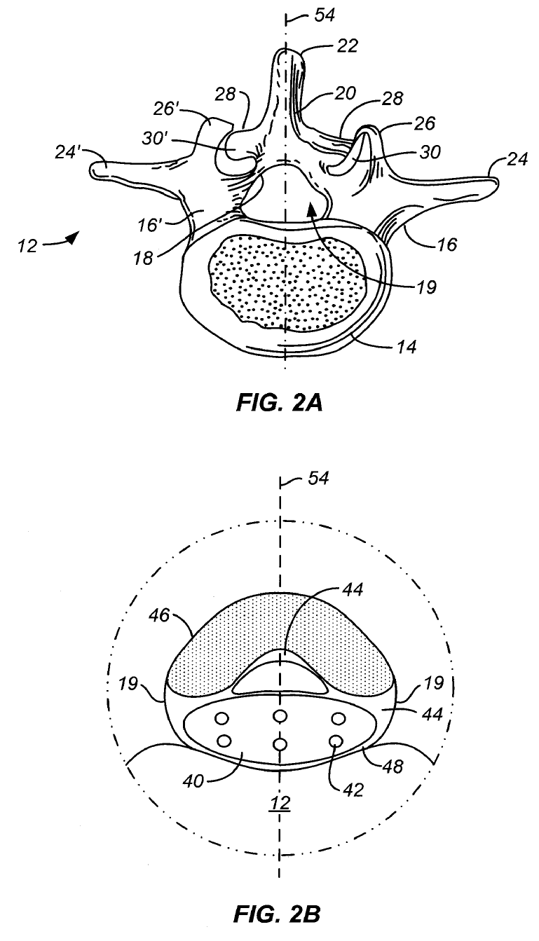

As discussed above, the devices and their usefulness can be illustrated in the context of spinal pathologies. In order to appreciate the usefulness of the devices it is helpful to understand an anatomical environment where the devices can be used. Thus, for example, the devices are designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4. An example of one of the vertebra is illustrated in FIG. 2A which depicts a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles 16, 16′, extend dorsally from each side of the vertebral body 14 to form a vertebral arch 18 which defines the vertebral foramen 19 which houses the spinal cord and associated meninges. At the posterior end of each pedicle 16, the vertebral arch 18 flares out into broad plates of bone known as the laminae 20. The laminae 20 fuse with each other to form a spinous process 22. The spinous process 22 provides for muscle and ligamentous attachment. A smooth transition from the pedicles 16 to the laminae 20 is interrupted by the formation of a series of processes.

Two transverse processes 24, 24′ thrust out laterally, one on each side, from the junction of the pedicle 16 with the lamina 20. The transverse processes 24, 24′ serve as levers for the attachment of muscles to the vertebrae 12. Four articular processes, two superior 26, 26′ and two inferior 28, 28′, also rise from the junctions of the pedicles 16 and the laminae 20. The superior articular processes 26, 26′ are sharp oval plates of bone rising upward on each side of the vertebrae, while the inferior processes 28, 28′ are oval plates of bone that jut downward on each side. See also FIG. 4.

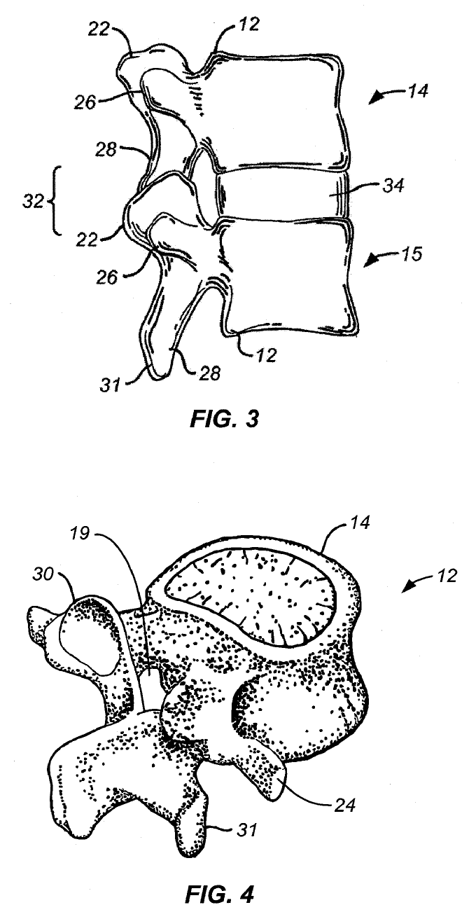

The superior and inferior articular processes 26 and 28 each have a natural bony structure known as a facet. The superior articular facet 30 faces medially upward, while the inferior articular facet 31 (see FIG. 3) faces laterally downward. When adjacent vertebrae 12 are aligned, the facets 30, 31, which are capped with a smooth articular cartilage and encapsulated by ligaments, interlock to form a facet joint 32.

An intervertebral disc 34 located between each adjacent vertebra 12 (with stacked vertebral bodies shown as 14, 15 in FIG. 3) permits gliding movement between the vertebrae 12. The structure and alignment of the vertebrae 12 thus permit a range of movement of the vertebrae 12 relative to each other. FIG. 4 illustrates a posterolateral oblique view of a vertebrae 12.

The spinal cord 40 is a long, thin, tubular bundle of nerves 42 that is an extension of the central nervous system from the brain. The spinal cord 40 is positioned in the vertebral foramen 19 and protected by the bony vertebral column that forms the spinal column 10. The main function of the spinal cord 40 is transmission of neural inputs between the periphery and the brain. Three meninges cover the spinal cord: the outer dura mater, the arachnoid mater and the innermost pia mater. Cerebrospinal fluid is found in the subarachnoid space and the spinal cord is stabilized within the dura mater by the connecting denticulate ligaments which extend from the enveloping pia mater between the dorsal and ventral roots. The lamina provides protection for the dural sac 48 and a foundation for the spinous processes. An epidural space 44 is provided between the spinal cord 40 and the vertebral arch 18 defining the vertebral foramen 19. A portion of the vertebral foramen 19 is also occupied by the ligamentum flavum 46. The ligamentum flavum 46 connects the lamina of adjacent vertebra 12. As discussed above, however, the ligamentum flavum 46 can become thickened, thereby reducing the cross-sectional volume in the vertebral foramen 19 available to house the spinal cord 40. As a result pressure is applied to the spinal cord 40 resulting in back pain, numbness of the legs, etc.

Thus, overall the spine 10 comprises a series of functional spinal units that are a motion segment surrounding the spinal cord 40 and which consist of two adjacent vertebral bodies 12, the intervertebral disc 34, associated ligaments, and facet joints 32. See Posner, I, et al. “A biomechanical analysis of the clinical stability of the lumbar and lumbosacral spine.” Spine 7:374-389 (1982).

Embodiments of the devices of the present invention include modular designs that are either or both configurable and adaptable. Additionally, the various embodiments disclosed herein may also be formed into a “kit” or system. As will be appreciated by those of skill in the art, as imaging technology improves, and mechanisms for interpreting the images (e.g., software tools) improve, patient specific adaptations of the tools and devices employing these concepts may be configured or manufactured prior to the surgery. Thus, it is within the scope of the invention to provide for patient specific tools and devices with integrally formed components that are pre-configured.

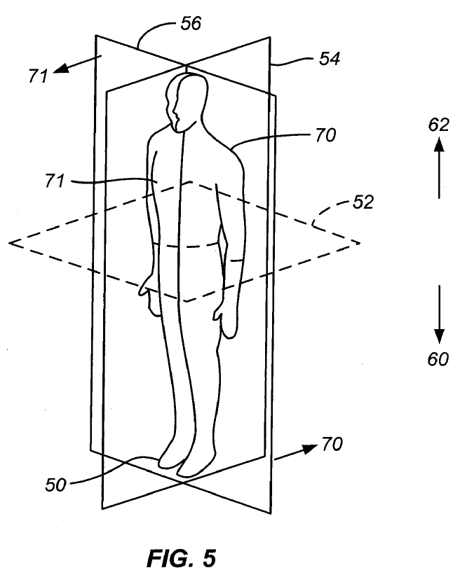

In order to understand the operational aspects of the invention, it is helpful to understand the anatomical references of the body 50 with respect to which the position and operation of the devices, and components thereof, are described. There are three anatomical planes generally used in anatomy to describe the human body 50 and structure within the human body: the axial plane 52, the sagittal plane 54 and the coronal plane 56 (see FIG. 5). Additionally, devices and the operation of devices are better understood with respect to the caudad 60 direction and/or the cephalad direction 62. Access to the body can be dorsally 70 (or posteriorly) such that the placement, operation or movement of the devices and tools is toward the back or rear of the body. Alternatively, devices and tools can be ventrally 71 (or anteriorly) such that the placement, operation or movement of the devices and tools is toward the front of the body. Various embodiments of the tools and systems of the present invention may be configurable and variable with respect to a single anatomical plane or with respect to two or more anatomical planes. For example, a tool or component thereof may be described as lying within and having adaptability in relation to a single plane. Similarly, the various components can incorporate differing sizes and/or shapes in order to accommodate differing patient sizes.

The vertebral column (spine, spinal column, backbone) forms the main part of the axial skeleton, provides a strong yet flexible support for the head and body, and protects the spinal cord disposed in the vertebral canal, which is formed within the vertebral column. The vertebral column comprises a stack of vertebrae, such as the two shown in FIG. 3, with an intervertebral disc between adjacent vertebrae. The vertebrae are stabilized by muscles and ligaments that hold the vertebrae in place and limit the movements of the vertebrae.

Referring back to FIGS. 2A and 3, each vertebra 12 includes a vertebral body 14 that supports a vertebral arch 18. A medial or saggital plane (54 in FIG. 5) generally divides vertebra 12 into two substantially equal lateral sides. The vertebral body 14 has the general shape of a short cylinder and is anterior to the vertebral arch 18. The vertebral arch 18 together with vertebral body 14 encloses a space termed the vertebral foramen 19. The succession of vertebral foramen 19 in adjacent vertebrae 12 along the vertebral column define the vertebral canal (spinal canal), which contains the spinal cord 40.

Vertebral arch 18 is formed by two pedicles 16, 16′ which project posteriorly to meet two laminae 20. The two laminae 20 meet dorsal-medially to form the spinous process 22. At the junction of pedicles 16, 16′ and laminae 20, six processes arise. Two transverse processes 24, 24′ project dorsal and lateral, two superior articular processes 26, 26′ project generally superiorly and are positioned superior to two inferior articular processes 28, 28′ that generally project inferiorly.

The vertebral foramen 19 is generally an oval shaped space that contains and protects the spinal cord 40. Spinal cord 40 comprises a plurality of nerves 42 surrounded by cerebrospinal fluid (CSF) and an outermost sheath/membrane called the dural sac 48. The CSF filled dural sac 48 containing nerves 42 is relatively compressible. Posterior to the spinal cord 40 within vertebral foramen 19 is the ligamentum flavum 46. Laminae 20 of adjacent vertebral arches 18 in the vertebral column are joined by the relatively broad, elastic ligamentum flavum 46.

The vertebral foramen 19 contains a portion of the ligamentum flavum 46, the spinal cord 40, and an epidural space 44 between the ligamentum flavum 46 and the spinal cord 40. The spinal cord 40 comprises a plurality of nerves 42 surrounded by cerebrospinal fluid (CSF) contained within dural sac 48. Nerves 42 normally comprise only a small proportion of the dural sac 48 volume. Thus, the CSF filled dural sac 48 is somewhat locally compressible, as localized pressure causes the CSF to flow to adjacent portions of the dural sac. Epidural space 44 is typically filled with blood vessels and fat. The posterior border of the normal epidural space 44 generally defined by the ligamentum flavum 46, which is shown in its normal, non-thickened state in FIG. 2B.

FIG. 2B illustrates a case of spinal stenosis resulting from a thickened ligamentum flavum 46. Since vertebral foramen 19 is defined and surrounded by relatively rigid bone, its volume is essentially constant. Thus, thickening of the ligamentum flavum 46 within the vertebral foramen 19 can eventually result in compression of the spinal cord 40. In particular, the thickened ligamentum flavum 46 may exert a compressive force on the posterior surface of dural sac 48. In addition, thickening of the ligamentum flavum 46 may compress the blood vessels and fat occupying the epidural space 44.

Compression of the spinal cord 40, particularly in the lumbar region, may result in low back pain as well as pain or abnormal sensations in the legs. Further, compression of the blood vessels in the epidural space 44 that houses the nerves of the cauda equina may result in ischemic pain termed spinal claudication.

In order to relieve the symptoms associated with a thickened or enlarged ligamentum flavum 46, methods, techniques, and devices described herein may be employed to reduce the compressive forces exerted by the thickened ligamentum flavum on the spinal cord 40 and the blood vessels in epidural space 44 (e.g., decompress spinal cord 40 and blood vessels in epidural space 44). Compressive forces exerted by the thickened/enlarged ligamentum flavum 46 may be reduced by a ligament decompression procedures described herein. The ligament decompression procedure is generally minimally invasive which provides benefits that will be appreciated by those skilled in the art. The ligament decompression procedure can reduce the size of the ligamentum flavum 46 by excising portions of the ligamentum flavum 46. In some embodiments, the ligament decompression procedure may be performed percutaneously. In some embodiments of the ligament decompression procedure, the ligamentum flavum 46 is reduced using an ipsilateral approach of the ligament decompression procedure. Using this approach, the ligamentum flavum 46 can be accessed from the ipsilateral side, or the same side, of the vertebral arch 18. The ligamentum flavum 46 can then be cut and removed ipsilaterally by a percutaneous cranial-caudal approach.

### II. Tissue Separation Devices

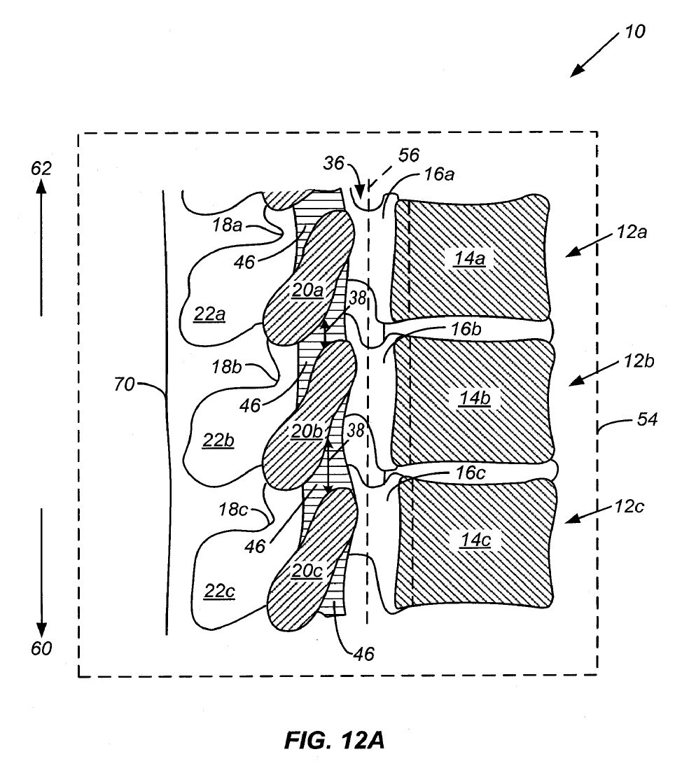

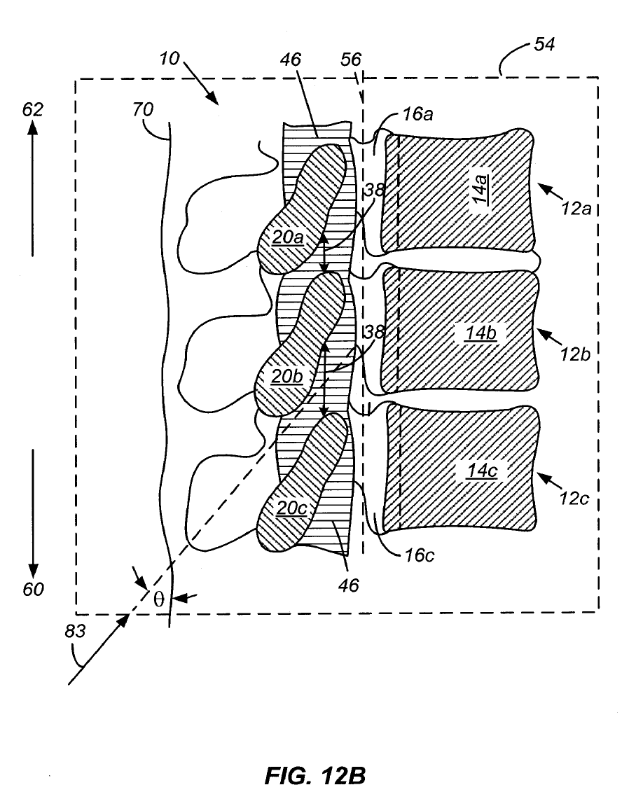

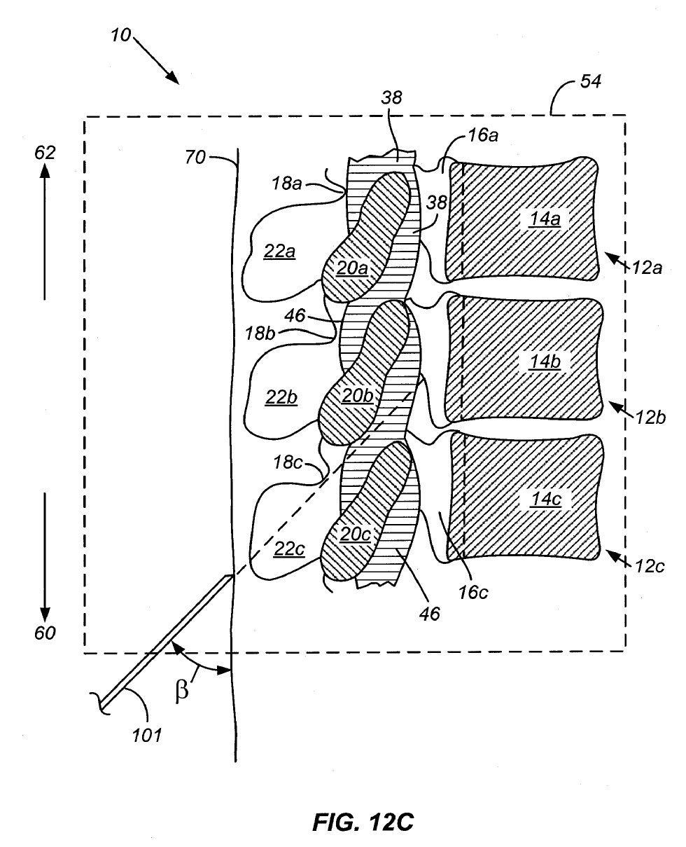

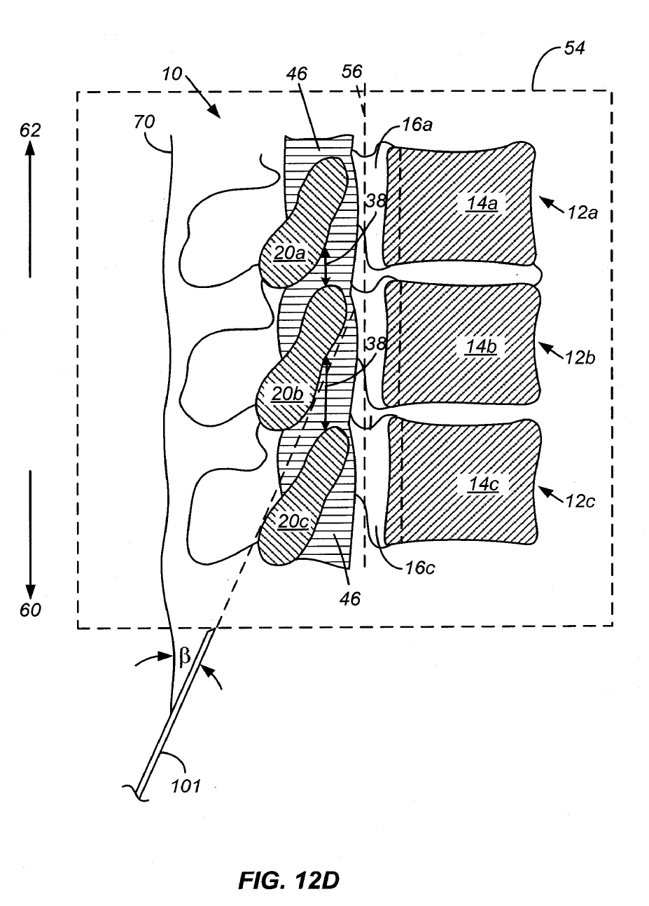

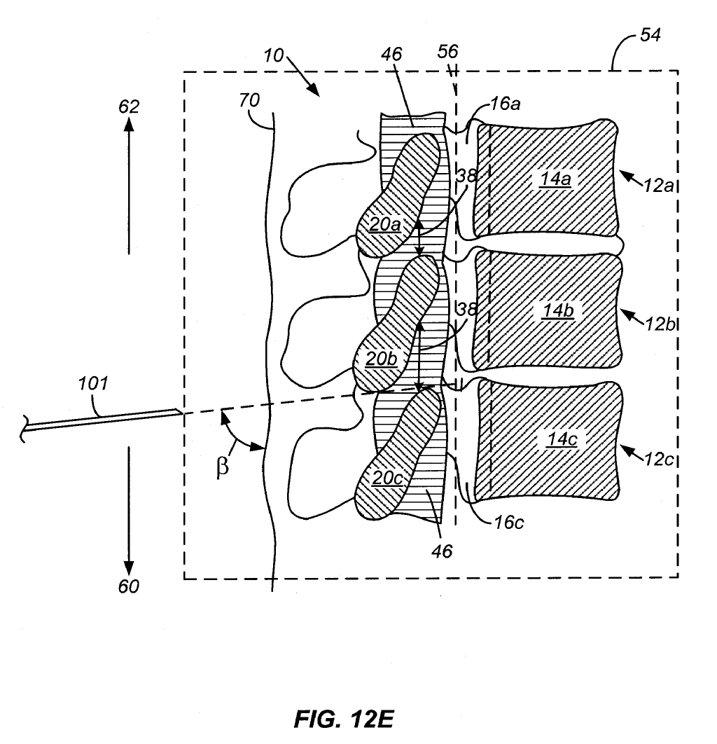

Embodiments of tissue separation tools, devices, and methods disclosed herein may take several forms and may be used according to an ipsilateral approach for minimally invasive ligament decompression procedure method described below, or used according to alternative minimally invasive ligament decompression procedures (e.g., minimally invasive ligament decompression procedure illustrated in FIG. 12). One such alternative minimally invasive ligament decompression procedure is disclosed in US 2006/0036272 which published on Feb. 16, 2006. In the descriptions of the tissue separation devices below, the distal portions of the devices are described in detail. As will be appreciated by those skilled in the art, distal refers to positions that are relatively closer to the region of interest (e.g., the thickened portion of the ligamentum flavum 46 to be decompressed) and farthest from the point of attachment or use. In contrast, proximal refers to positions that are relatively close to point of attachment or use and furthest from the region of interest (e.g., the handle).

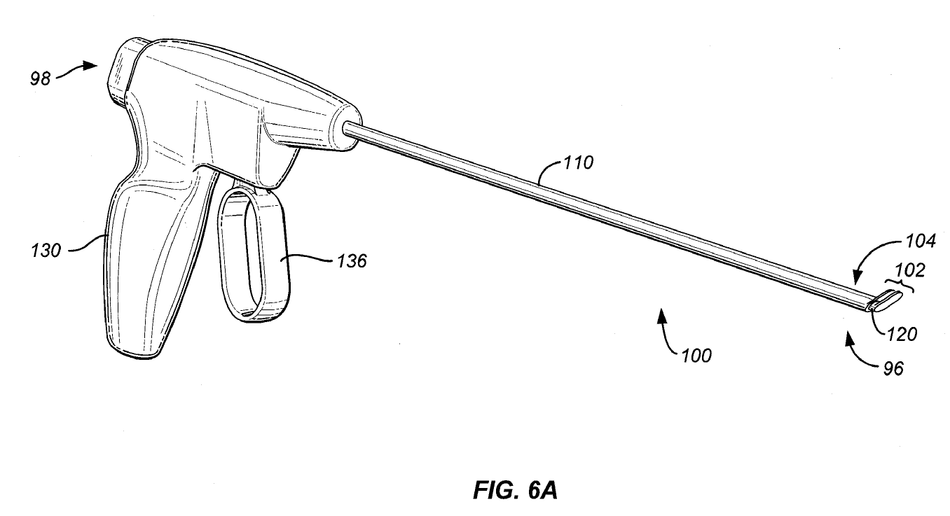

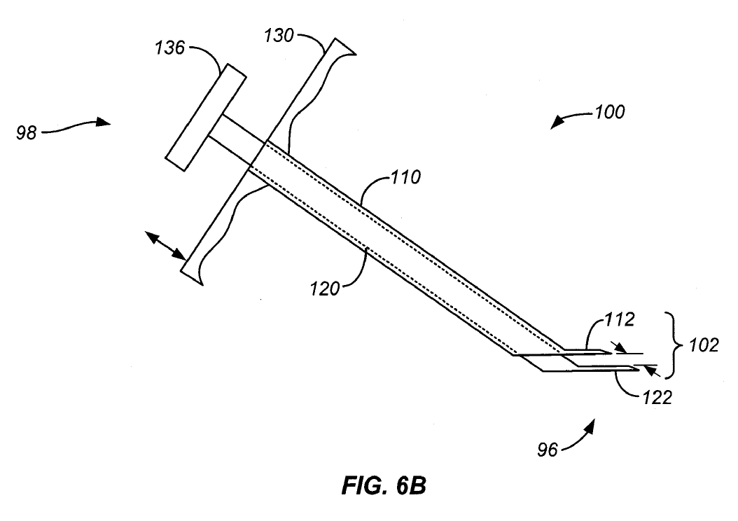

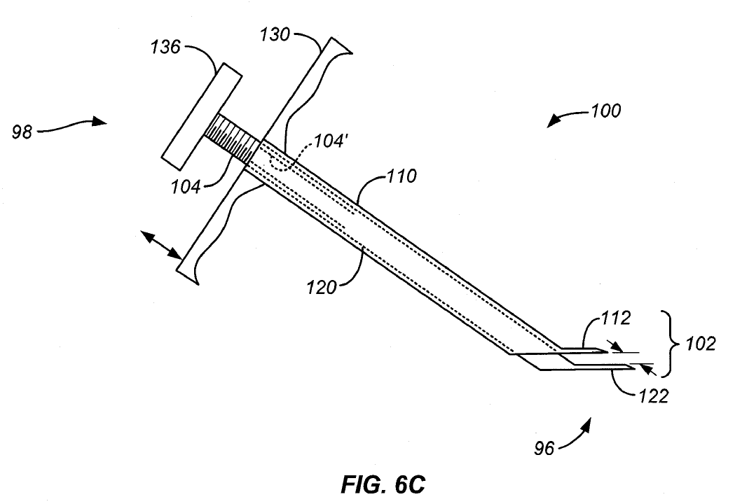

Referring now to FIG. 6A, an embodiment of a device 100 for separating targeted tissue comprises an elongate hollow body 110. Elongate inner body 120 is disclosed coaxially within hollow body 110 of device 100 to form tissue separation member 102. A handle 130 is provided which is adaptable and configurable to a user to control the operation of the distal 96 end of the device 100 from its proximal 98 end and an actuator 136, or trigger, can be provided to actuate operation of the device.

Referring now to FIG. 6B, elongate inner body 120 is disposed coaxially within the hollow body 110 and may be advanced or withdrawn using a variety of manual, automatic and semi-automatic mechanisms. In one example, inner member 120 may be coupled to a trigger or actuator 136, such as an outer handle, which may be proximal to handle 130. Thus, in one embodiment, handle 130 may be moved while outer handle 136 remains stationary. Alternatively, handle 130 may stay stationary while the outer handle 136 moves.

As shown in FIG. 6C, the proximal end of the device 100 may be adapted to incorporate a threaded mechanism on the exterior surface 104 of at least a proximal portion of the elongate inner body 120 and a corresponding threaded mechanisms on the interior surface 104′ of at least a portion of a proximal portion of the elongate outer body 110. The complementary threaded mechanisms can be calibrated to more precisely move at least one of inner member 120 and distal separation member 122 in a proximal or distal direction to facilitate separation of the components of the tissue separation member 102.

It also contemplated that a ratcheting mechanism may be used to move lower or distal separation member or spreader 122 and/or upper or proximal separation member or spreader 112 incrementally apart or together.

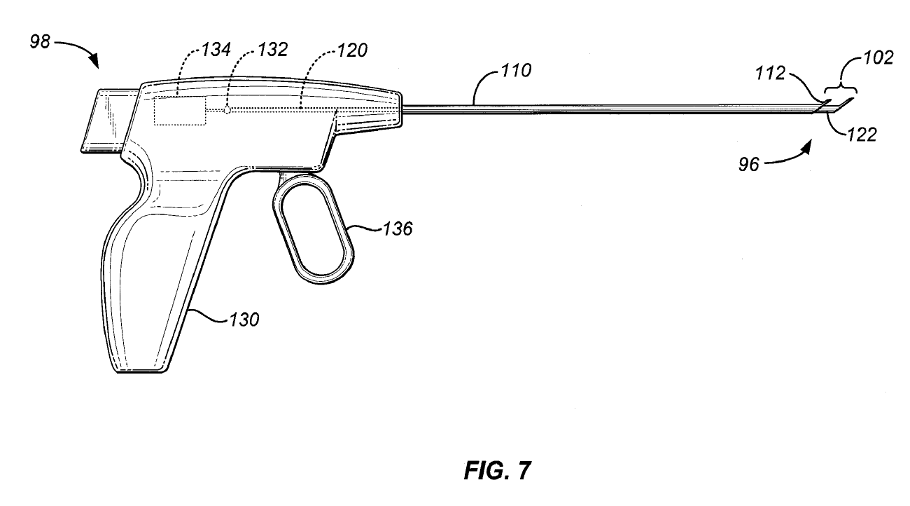

As shown in FIG. 7, a drive member 132 can be provided that is coupled to a motor 134 that automatically or semi-automatically controls member 132, thereby actuating one or both of the proximal separation member 112 and the distal separation member 122 of the tissue separation member 102. Drive member 132 may be mechanically, electrically, or electromechanically coupled to tissue separation member 102 by any suitable mechanism including, for example, gears, frictional engagement, belts, or combinations thereof to transfer for example, a rotational torque provided by motor 134 to tissue separation member 102. In this embodiment, motor 134 is enclosed in a handle 130 that is coupled to elongate hollow body 110. Hollow body 110 may be releasably coupled to handle 130, thereby permitting periodic access to elongate hollow body 110.

In general, motor 134 may comprise any suitable device adaptable or configurable to drive the rotation of a drive member 132 and tissue separation member 102 including, without limitation, an electric motor, a hydraulic motor, a pneumatic motor, and the like. Motor 134 preferably comprises an electrical motor. In such embodiments, motor 134 may be powered by a rechargeable battery (e.g., lithium ion batteries, nickel cadmium batteries, etc.) or with electricity provided from a conventional outlet. In this embodiment, motor 134 is switched on and off via a trigger 136 operable by a finger of the individual using device 100. In other embodiments, motor 134 may be switched on and off with a switch provided on the handle (e.g., handle 130).

In this embodiment, hollow body 110 is an elongate cylindrical tubular having a circular cross-section. Suitable cross-sections range, for example, from 0.1 inch to 0.5 inches. However, in general, hollow body 110 may have any suitable cross-sectional shape including, without limitation, hexagonal, rectangular, etc. Hollow body 110 may be rigid, flexible or variably rigid and flexible along its length, or adaptable and configurable to have a first configuration, e.g., rigid, and then a second configuration, e.g., flexible, as needed. Examples of suitable hollow bodies include without limitation, cannulas, hypotubes, catheters, and the like. Furthermore, hollow body 110 may optionally be sized to be disposed coaxially within a guiding catheter.

Although hollow body 110 is depicted to be a straight, elongate body, it is contemplated that hollow body 110 may be articulated as desired to separate a targeted tissue requiring a tortuous access path. That is, hollow body 110 may comprise suitable angles to permit advancement through a non-linear path to the targeted tissue. Alternatively, hollow body 110 may have a smoothly curved configuration analogous to the path to be taken to access the targeted tissue.

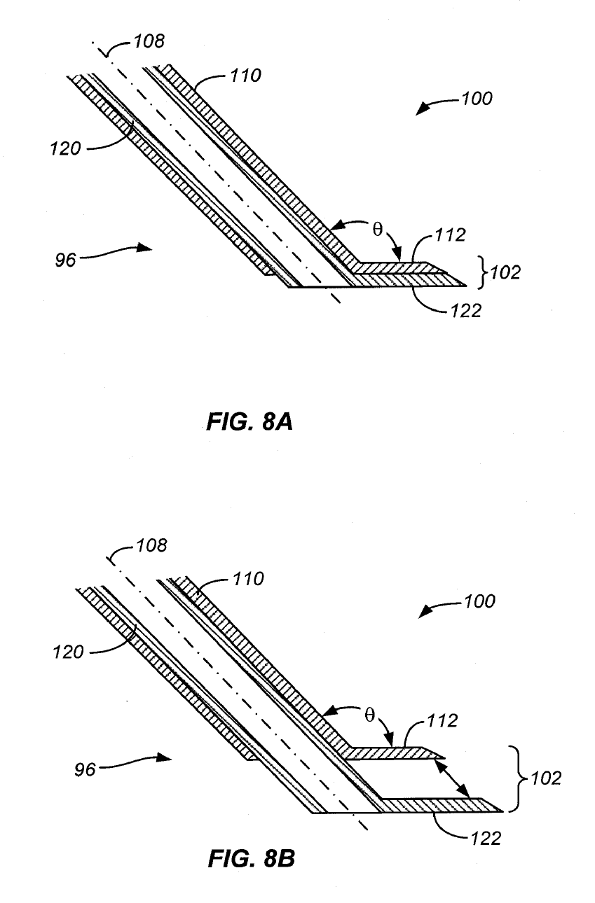

Referring now to FIGS. 8A-B, an embodiment of a device 100 for separating a tissue, such as a ligament, from another anatomical structure (e.g., bone, a different ligament, fat, etc.) is shown. Device 100 comprises an elongate hollow body 110 having a central longitudinal axis 108 and an elongate inner body 120 coaxially disposed within outer hollow body 110. Generally, the elongate inner body has a length that is greater than its width or diameter. The distal end 96 of outer hollow body 110 includes an opening or aperture through which inner body 120 extends. The elongate inner body 120 can be hollow or solid.

Outer elongate body 110 further comprises a proximal separation member 112 that extends laterally from the outer surface of outer body 110 at distal end 96. In this embodiment, separation member 112 is substantially planar, however, in other embodiments, it may be non-planar (e.g., curved), or any other configuration that facilitates separation of a target tissue. Separation member 112 is oriented at an angle θ measured relative to the longitudinal axis 108 of elongate body 110.

Elongate inner body 120 further comprises a distal or lower separation member 122 that extends laterally from the outer surface of body 120 also at its distal end 96. In this embodiment, separation member 122 is substantially planar, and is disposed substantially parallel to upper separation member 112. However, in other embodiments, separation member 122 may be non-planar (e.g., curved) and/or non-parallel with upper separation member 112. Separation member 122 is also oriented at angle θ measured relative to central longitudinal axis 108.

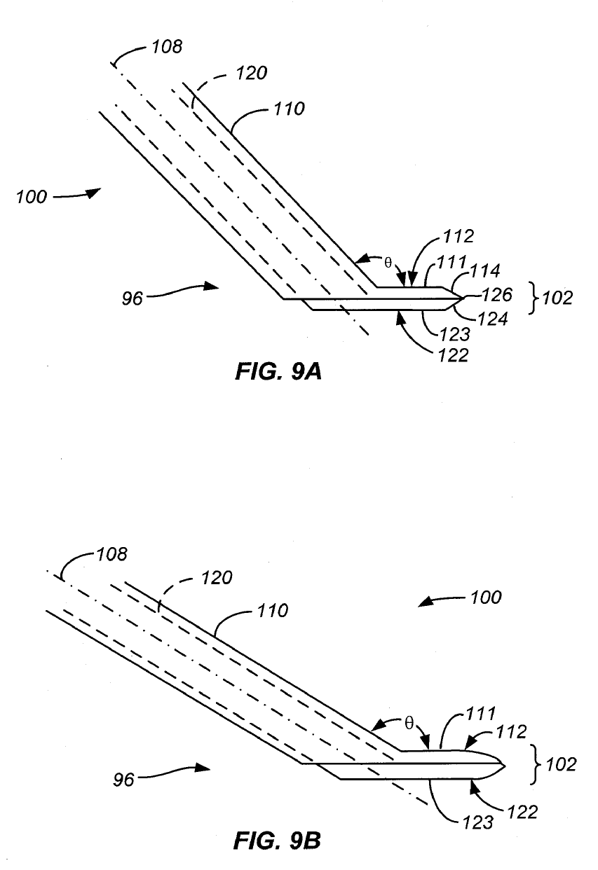

Referring still to FIGS. 8A and 8B, inner body 120 is slidingly disposed within outer hollow body 110 such that bodies 120, 110 may move axially relative to each other. The outer radius of inner body 120 is preferably substantially the same or slightly less than the inner radius of outer body 110, such that inner surface of outer body 110 slidingly engages the outer surface of inner body 120. In such embodiments, the engagement of inner surface of outer body 110 with the outer surface of inner body 120 restricts relative radial movement therebetween. As a result of the relative axial movement between bodies 110, 120, lower separation member 122 is permitted to move axially relative to upper separation member 112. More specifically, in the closed position of FIG. 8A, separation members 112, 122 are generally disposed adjacent each other. However, in the opened position of FIG. 8B, separation members 112, 122 are axially spaced apart. With members 112, 122 wedged between a tissue and another anatomical structure in the closed position (FIG. 9), and then moved axially apart by moving inner body 120 axially relative to outer body 110, the tissue may be spaced apart from the other anatomical structure. The movement members 112, 122 relative to each other may be performed manually or by an actuator (not shown). Moreover the devices can be configured such that the proximal separation member 112 moves while the distal separation member 122 remains stationary, the proximal separation member 112 remains stationary while the distal separation member 122 moves, or both the proximal and distal separation members are capable of concurrent or serial movement.

In the embodiment shown in FIGS. 9A and 9B, outer hollow body 110 is an elongate cylindrical tubular having a circular cross-section. However, in general, outer hollow body 110 may comprise any suitable cross-sectional shape including, without limitation, hexagonal, rectangular, etc. Examples of suitable hollow bodies include without limitation, cannulas, hypotubes, catheters, and the like. Further, outer hollow body 110 may be rigid or flexible as desired, or may have rigid sections and flexible sections along its length. In some embodiments, device 100 may be advanced to the region of interest via a guiding catheter.

Inner body 120 may either be hollow or solid. As with outer hollow body 110, inner body 120 may have any suitable cross-sectional shape including, without limitation, hexagonal, rectangular, etc. Inner body 120 may be made of the same material or a different material as outer hollow body 110. Preferably, inner body 120 is made of a flexible material.

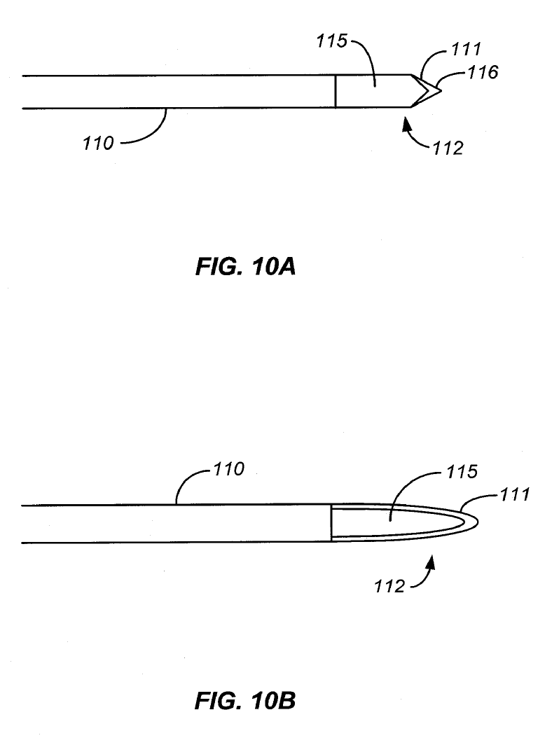

As discussed above, outer hollow body 110 and inner body 120 have an upper separation member 112 and a lower separation member 122, respectively for facilitating the separation of a tissue from another anatomical structure (e.g., bone). When viewed in profile, upper and lower separation members 112, 122, may be configured in any number of geometrical configurations. As shown in FIG. 9A, outer face 111 of upper members 110, and outer face 123 of inner member 120 are flat or planar and have beveled outer edges 114, 124 to facilitate insertion of the device 100 between tissue and structure to be spaced from the ligament. Beveled edges 114, 124 may either be alternatively angled to form a point as show in FIG. 9A, or may be angled to form a slope as seen in FIG. 9B. Alternatively, upper and lower members 112, 122 may have curved convex surfaces as shown in FIGS. 9B and 10B. Separation members 112, 122 can be configured to have a concave configuration for assisting in separation of ligaments and/or bone. Moreover, separation members 112, 122 can be symmetric in side view as seen in FIG. 9A-B. However, it is contemplated that some embodiments of device 100 may have asymmetric separation member 112, 122. For example, upper separation member 112 may have a flat outer surface 111 while outer face 123 of lower separation member may have a convex, curved surface, and vice versa.

As previously described, both upper (proximal) and lower (distal) separation members 112, 122 are disposed at an angle θ relative to central longitudinal axis 108. Angle 0 is preferably an obtuse angle (i. e. greater than about 90 degrees). More specifically, angle θ preferably ranges from about 90 degrees to about 175 degrees, and more preferably ranges from about 110 degrees to about 150 degrees. Upper and lower separation members 112, 122 may be integral with bodies 110, 120, respectively, or coupled to bodies 110, 120, respectively. In some embodiments, one or both of members 112, 122 may be coupled to bodies 110, 120, respectively, such that angle θ may be adjusted as needed. In the open configuration shown in FIG. 8B, upper and lower separation members 112, 122 may be adjusted to be open at any suitable axial distance apart. The distance between upper and lower separation members 112, 122 in the open configuration preferably ranges from about 3 mm to about 16.

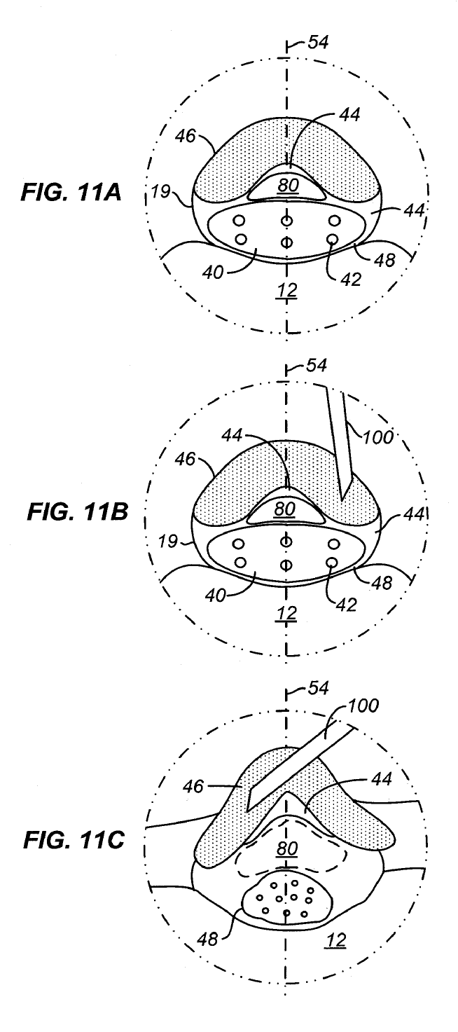

FIGS. 10A-B show the top view of different embodiments of the device 100. Outer edge 111 of upper separation member 112 and outer edge 121 (not shown) of lower separation member 122 (not shown) may be angled or tapered for better insertion of device between tissue and bone. In other embodiments, outer edge 111 and outer edge 121 (not shown) of upper separation member 112 and lower separation member 122 (not shown), respectively, may have a curved aspect as shown in FIG. 11B. Outer face 115 and outer face 125 (not shown) of upper separation member 112 and lower separation member 122 (not shown), respectively, may comprise a texture such as a knurling/diamond texture or a nodular texture to provide enhanced grip of tissue and/or bone. As will be appreciated by those skilled in the art, the outer edge 121 and outer face 125 are positioned beneath the outer edge 111 and outer face 115 in these figures.

Referring back to FIG. 6B, proximal end of outer hollow body may be coupled to inner handle. Inner member 120 may be advanced using a variety of mechanism. More particularly, inner member 120 may be coupled to an outer handle 136, which may be proximal to inner handle 130. Thus, inner handle 130 may be moved while outer handle 136 remains stationary. Alternatively, proximal end of device 100 may incorporate a threaded mechanism as described above to more precisely move inner member 120 and lower separation member 122 in a distal direction. It also contemplated that a ratcheting mechanism may be used to move lower separation member 122 and/or upper separation member 112 incrementally apart or together.

### III. Methods of Treatment

A. Creation of a Safety Zone

As shown in FIG. 2B, the ligamentum flavum 46 is posteriorly apposed to the spinal cord 40. The ligamentum flavum 46 can become enlarged. The placement of tools within the ligamentum flavum 46 to separate portions of the ligamentum flavum 46 creates a risk of inadvertent damage to the spinal cord 40, dural sac 48, and/or nerves 42. Thus, in some embodiments of the procedures described herein, prior to insertion of tissue separation devices into the ligamentum flavum 46, a gap, pocket, or space is advantageously created between the ligamentum flavum 46 and the spinal cord 40 to provide a safety zone 80 (illustrated in FIG. 11) between the ligamentum flavum 46 and the spinal cord.

The vertebral foramen 19 includes the epidural space 44 and spinal cord 40 containing nerves 42 and CSF within the dural sac 48. Further, a thickened/enlarged ligamentum flavum 46 can, as will be appreciated by those skilled in the art, extend into the vertebral foramen 19. To reduce the risk of damage to the dural sac 48 and the spinal cord 40, a safety zone is created between the ligamentum flavum 46 and the dural sac 48 according to the methods disclosed herein.

As previously described, the spinal cord 40 comprises nerves 42 surrounded by CSF, and is contained within the dural sac 48. Since more than 90% of the volume of the dural sac 48 in the lumbar region is filled by CSF, the dural sac 48 is highly compressible. Thus, even when stenosis is causing compression of the spinal cord 40, in most cases it is possible to temporarily compress spinal cord 40 even further. The dural sac 48 can be further compressed in the region of interest by introducing a media into the epidural space 44 to create a safety zone. For example, the media can be a fluid, a gel, or any other suitable media for compressing the spinal cord. The media can be introduced into the epidural space 44 with an insertion member, such as a needle, catheter, cannula, or any other suitable insertion device. The media located in the safety zone gently applies an additional compressive force to the outer surface of the dural sac 48 so that at least a portion of the CSF within dural sac 48 is forced out of the dural sac 48 in the region of interest, resulting in a safety zone between the dural sac 48 and the ligamentum flavum 46.

According to some embodiments, the dural sac 48 can be compressed by introducing, for example, a contrast medium into the region of interest in the epidural space 44. The introduction of the contrast medium can provide contrast guided dural protection. Additionally, the contrast medium can be used to create a safety zone or to aid in the visualization of the surgical area. In some embodiments, the contrast medium can be used to both create the safety zone and to aid in imaging the region of interest. The contrast medium can be a standard radio-opaque non-ionic myelographic contrast medium or any other suitable imagable or non-imagable contrast medium. The contrast medium can be introduced into the epidural space by injection of the contrast medium. In some embodiment, the injection is a percutaneous injection. A sufficient amount of contrast media can be injected into the region of interest in the epidural space 44 to displace the CSF out of the region of interest and to compress the dural sac 48. The material can compress the dural sac 48 entirely. Alternatively, the material can compress the dural sac 48 partially. The dural sac 48 can be compressed to any desired degree. Once introduced into the region of interest, the introduced media can be entirely contained within the confines of the epidural space 44. At the same time, the media extends to the margins of the dural sac 48. Alternatively, the introduced media can be partially contained within the confines of the epidural space 44. The epidural space 44 is substantially watertight and the fatty tissues and vascularization in epidural space 44, combined with the viscous properties of the contrast medium, serve to substantially maintain the injected medium in the desired region of interest.

Once a safety zone 80 has been created, a tissue separation tool or device 100, such as those described above, may be inserted into the ligamentum flavum 46. Device 100 may comprise any suitable device, tool or instrument for relieving stenosis caused by the thickened/enlarged ligamentum flavum 46 including without limitation, embodiments of tissue separation devices and tissue retraction devices described herein. In some embodiments, device 100 is inserted and positioned in the ligamentum flavum 46 on the same side (ipsilateral) of the sagittal plane 54 as device 100 percutaneously accesses the body, such that device 100 does not cross the sagittal plane 54 as shown in FIG. 11B. Alternatively, device 100 can be positioned in the ligamentum flavum 46 on the opposite side of sagittal plane 54 as device 100 percutaneously accesses the body, such that device 100 crosses the sagittal plane 54 as shown in FIG. 11C. In some embodiments, the tissue separation device 100 can be guided by and advanced through a cannula toward the ligamentum flavum 46. In some embodiments, the device 100 can be advanced toward the ligamentum flavum 46 without the use of a cannula.

While it is preferred that the tip of device 100 remain within the ligamentum flavum 46 as shown, the presence of the safety zone reduces the likelihood that the dural sac 48 will be damaged, even if the tip of device 100 breaks through the anterior surface of the ligamentum flavum 46.

Because the present techniques are preferably performed percutaneously, certain aspects of the present disclosure may be facilitated by imaging. Imaging windows (e.g., a fluoroscopic window of access) can be employed to aid in performance of all or part of the procedures described herein. For instance, an imaging window can be employed to aid in insertion of device 100 into the ligamentum flavum 46.

The spine can be imaged using any suitable technology including, without limitation, 2D fluoroscopy, 3D fluoroscopy, CT, MRI, and ultrasound. The spine can also be directly visualized using fiber optic or microsurgical techniques. Stereotactic or computerized image fusion techniques are also suitable for imaging the spine. Fluoroscopy is currently particularly well-suited to the techniques disclosed herein. Fluoroscopic equipment is safe and easy to use, readily available in most medical facilities, and relatively inexpensive. In a typical procedure, using direct biplane fluoroscopic guidance and local anesthesia, the epidural space 44 is accessed for injection of contrast media adjacent to the surgical site. In some instances, the procedure can be performed without imaging the spine in advance.

If the injected medium is radio-opaque, as are for example myelographic contrast media, the margins of the expanded epidural space 44 will be readily visible using fluoroscopy or CT imaging. Thus, the safety zone created by the present contrast-guided dural compression techniques can reduce the risk of damage to the dural sac 48 and the spinal cord 40 during ligament decompression procedures to remove or displace portions of the ligamentum flavum 46 and/or laminae 20 in order to treat spinal stenosis.

B. Use of Injectable Medium

In one aspect of the invention, the medium introduced into the epidural space 44 can be a gel, including, but not limited, to a re-sorbable water-soluble gel. A gel can be used to localize the safety zone 80 at the site of surgery and to reduce leakage of the contrast medium from the protective layer from the vertebral/spinal canal 36. In some embodiments, the contrast medium can be an injectable gel. The gel can be more viscous than conventional contrast media. The viscosity of the gel enables the gel to be localized at the desired region of interest. This is in contrast to standard liquid contrast media that are used in epidurography, which have more of a tendency to spread out from the region where injected. The use of a gel can result in more uniform compression of the dural sac 48 and less leakage of contrast medium out of the vertebral/spinal canal. In addition, contrast gels can be more slowly re-absorbed allowing for better visualization of the region of interest during the entire course of the surgical procedure. In some embodiments, an amount of gel is introduced as is necessary to compress the dural sac a desired amount. In some embodiments, an expandable gel is introduced. The gel can expand to fill the epidural space and to compress the dural sac. In some embodiments, the gel is re-absorbed at a rate allowing for better visualization of the region of interest during part of the surgical procedure.

A gel can be introduced into the epidural space 44. The gel can either comprise a contrast agent or a contrast agent can be introduced in the epidural space 44 simultaneously with the gel. An amount of contrast agent can be introduced into the epidural space followed by an amount of gel. Alternatively, an amount of gel can be introduced into the epidural space followed by an amount of contrast agent. The contrast agent can be captured on the surface of the gel mass, so that the periphery of the gel mass is imagable.

In some embodiments, a copolymer gel can be used including, but not limited to, standard hydrophilic-lipophilic block copolymer gel, or any other suitable gel can be used. In some embodiments, the gel comprises an inert base. The gel material can be a temperature dependent gel material. In some embodiments, the gel can be a liquid at ambient temperatures and can be injected into the epidural space through a small bore, such as a 27 gauge needle. When warmed to body temperature, the gel can thicken thereby becoming more viscous. The viscosity of the gel can also be adjusted through the specifics of the preparation of the gel. In some embodiments, the injected gel attains a viscosity that is two, three, six or even ten times that of the fluids that are typically used for epidurograms. The gel or other fluid can be sufficiently viscid or viscous at body temperature to compress and protect dural sac 48 in the manner described above. In some embodiments, the gel can remain in the region of interest for at least about thirty (30) minutes after being injected into the epidural space.

In certain embodiments, the injected medium undergoes a reversible change in viscosity when warmed to body temperature so that it can be injected as a low-viscosity fluid, thicken upon injection into the patient, and be returned to its low-viscosity state by cooling. In some embodiments, the injected medium is introduced to the epidural space as desired and the gel thickens upon being warmed by the body temperature. In some embodiments, the gel can be removed by contacting the gel with a heat removal device, such as an aspirator that has been provided with a cooling tip, needle, catheter, or other suitable cooling device. As a result of localized cooling, the gel can revert to its initial non viscous liquid state and can be easily suctioned up by the aspirator, or other suitable suction device.

An example of a suitable contrast medium having the desired properties is OMNIPAQUE® 240 (iohexol) available from Nycomed, N.Y., which is a commercially available non-ionic iodinated myelographic contrast medium. Other suitable media will be known to those skilled in the art. Because of the proximity to the spinal cord 40 and spinal nerves 42, it is preferred not to use ionic media in the media. In some embodiments, the compositions are reabsorbed relatively rapidly after the procedure and any residual compression on the dural sac 48 after the ligament decompression procedure dissipates relatively quickly. For example, in some embodiments, the gel can have sufficient viscosity to compress the dural sac 48 for thirty (30) minutes, and sufficient degradability to be substantially reabsorbed within approximately two hours.

The introduced contrast medium can further comprise one or more bioactive agents. For example, medications such as those used in epidural steroid injections (e.g., Depo Medrol® (methylprednisolone acetate), Celestone® Soluspan® (betamethasone sodium phosphate and betamethasone acetate) can be added to the epidural gel to speed healing and reduce inflammation, scarring, and adhesions. The gel can release the steroid medication slowly and prolong the anti-inflammatory effect, which can be extremely advantageous. Local anesthetic agents can also be added to the gel. This prolongs the duration of action of local anesthetic agents in the epidural space to prolong pain relief during epidural anesthesia. In this embodiment the gel can be formulated to slow the re-absorption of the gel.

The gels can also be used for epidural steroid injection and perineural blocks for management of acute and chronic spinal pain. Thrombin or other haemostatic agents can be added if desired, so as to reduce the risk of bleeding.

In some embodiments, the gel can also be used as a substitute for a blood patch if a CSF leak occurs. The gel can also be used as an alternative method to treat lumbar puncture complications such as post-lumbar puncture CSF leak or other causes of intracranial hypotension. Similarly, the gel can be used to patch postoperative CSF leaks or dural tears. If the dural sac is inadvertently torn or cut, the gel can serve to immediately seal the site and prevent leakage of the cerebral spinal fluid.

C. Imaging Techniques

As will be appreciated by those of skill in the art, imaging techniques suitable for assessing spinal stenosis and evaluating the epidural space include the use of x-rays, magnetic resonance imaging (MRI), computed tomography scanning (CT, also known as computerized axial tomography or CAT), optical coherence tomography, SPECT, PET, ultrasound imaging techniques, and optical imaging techniques. (See, also, U.S. Pat. No. 6,373,250 to Tsoref et al.; and Vandeberg et al. (2002) Radiology 222:430-436). Contrast or other enhancing agents can be used using any route of administration, e.g. intravenous, intra-articular, etc.

In certain embodiments, CT or MRI is used to assess tissue, bone, cartilage and any defects therein, and to provide morphologic or biochemical or biomechanical information about the target area of interest. For discussions of the basic NMR principles and techniques, see MRI Basic Principles and Applications, Second Edition, Mark A. Brown and Richard C. Semelka, Wiley-Liss, Inc. (1999). Two-dimensional, three-dimensional images, or maps, of the target area alone or in combination with a movement pattern, e.g. flexion-extension, translation and/or rotation, can be obtained. Three-dimensional images can include information on movement patterns, contact points, contact zone of two or more opposing surfaces, and movement of the contact point or zone during joint motion. Two and three-dimensional images can include information on biochemical composition of the articular cartilage. In addition, imaging techniques can be compared over time, for example to provide up-to-date information on the shape and type of repair material needed.

Any of the imaging devices described herein can also be used intra-operatively, for example using a hand-held ultrasound and/or optical probe to image the target area intra-operatively.

D. Approaching the Stenosis Ipsilaterally for Minimally Invasive Ligament Decompression Procedures

Once the safety zone 80 has been created, the margins of the epidural space 44 are clearly demarcated by the introduced medium and can be visualized radiographically if an imageable medium has been used. As mentioned above, percutaneous procedures can be performed more safely on the ligamentum flavum 46 and/or surrounding tissues while reducing the potential for injuring the dural sac 48 and the spinal cord 40. As shown in FIGS. 11A-C, and discussed above, the ligamentum flavum 46 can be accessed ipsilaterally or contralaterally.

A variety of suitable techniques and devices may be employed to reduce the size of the thickened/enlarged ligamentum flavum 46, thereby decompressing the spinal cord 40 as well as blood vessels contained within the epidural space 44. Examples of suitable decompression techniques include without limitation, removal of tissue from the ligamentum flavum 46, laminectomy, laminotomy, and retraction and anchoring of the ligamentum flavum 46. In some embodiments, all or a portion of the ligamentum flavum 46 is separated using a tissue separation device or tool (e.g., devices 100 described herein).

Accessing the ligamentum flavum 46 with one of the tissue separation devices 100 to remove portions of the ligamentum flavum 46 can present significant challenges. For instance, in some conventional approaches to correct stenosis caused by an enlarged ligamentum flavum 46, an incision is made in the back of the patient and then the muscles and supporting structures of the vertebral column (spine) are stripped away, exposing the posterior aspect of the vertebral column. Subsequently, the thickened ligamentum flavum 46 is exposed by removal of a portion of the vertebral arch 18, often at lamina 20, which encloses the anterior portion of the spinal canal (laminectomy). The thickened ligamentum flavum 46 can then be excised by sharp dissection, e.g., with a scalpel or punching instruments. However, this approach is usually performed under general anesthesia and typically requires an extended hospital stay, lengthy recovery time and significant rehabilitation. As another example, some ligament decompression procedures access the ligamentum flavum 46 percutaneously by boring a hole through the vertebral arch 18 of the vertebra 12, often through a lamina 20. A cannula and/or device 100 may be passed through the bore and/or anchored to the bore to access ligamentum flavum 46 for modification and/or separation. However, while such a ligament decompression procedure is minimally invasive and reduces recovery time, such an approach requires the additional step of boring a hole in the posterior of the vertebra 12 of interest. Thus, in some cases it will be preferable to employ a ligament decompression procedure that percutaneously accesses the ligamentum flavum 46 without the need to cut or bore through the vertebrae.

FIGS. 12A-E are a partial cross-sectional lateral view of a segment of a spinal column 10. The partial cross-sectional view is taken across a sagittal plane 54. The segment of spinal column 10 illustrated in FIG. 12A includes three vertebrae 12a, 12b, and 12c. Each vertebra 12a, 12b, 12c includes a vertebral body 14a, 14b, 14c, that supports a vertebral arch 18a, 18b, 18c, respectively. Vertebral body 14a, 14b, 14c is anterior to vertebral arch 18a, 18b, 18c, respectively. Each vertebral arch 18a, 18b, 18c together with vertebral body 14a, 14b, 14c, respectively, encloses a vertebral foramen. The succession of vertebral foramen in adjacent vertebrae 12a, 12b, 12c defines vertebral canal 36 (spinal canal) that runs along the length of vertebral column 10 and which is illustrated along the length of the intersection between the sagittal 54 and coronal 56 planes. Vertebral canal 36 contains the spinal cord (not shown in FIG. 12).

As previously described, each vertebral arch 18a, 18b, 18c includes two pedicles 16a, 16b, 16c, which project posteriorly to meet two lamina 20a, 20b, 20c, respectively. It is to be understood that in this view, one pedicle has been removed from each vertebra 12a, 12b, 12c and only the cross-section of one lamina 20a, 20b, 20c is visible. The two lamina 20a, 20b, 20c meet dorsal-medially to form the spinous process 22a, 22b, 22c, respectively.

Lamina 20a, 20b, 20c of adjacent vertebra 12a, 12b, 12c are connected by the ligamentum flavum 46 (shown in cross-section). The relatively elastic ligamentum flavum 46 extends almost vertically from the superior lamina to the inferior lamina of the adjacent vertebrae. In particular, the ligamentum flavum 46 originates on the inferior surface of the laminae of the superior vertebrae and connects to the superior surface of the laminae of the inferior vertebrae. The ligamentum flavum 46 originates on the inferior surface of lamina 20a of superior vertebra 12a and connects to the superior surface of lamina 20b of the inferior vertebra 12b. Thus, the ligamentum flavum 46 spans an interlaminar space 38 (i.e., space between laminae of adjacent vertebrae). The interlaminar space 38 is generally the space between laminae of adjacent vertebrae in the spinal column 10.

Still referring to FIGS. 12B-D, each lamina 20a, 20b, 20c comprises a relatively broad flat plate of bone that extends dorsal-medially and slightly inferiorly from pedicles 16a, 16b, 16c, respectively. Along the length of vertebral column 10, the lamina 20a, 20b, 20c overlap, with each lamina substantially parallel to and at least partially overlapping the adjacent inferior lamina. Further, the adjacent substantially parallel laminae are separated by the intervening ligamentum flavum 46 and the interlaminar space 38. For instance, the lamina 20a is substantially parallel to and partially overlaps adjacent inferior lamina 20b and is separated from lamina 20b by the ligamentum flavum 46 and the interlaminar space 38.

FIG. 12E illustrates vertebral column 10 as may be encountered during a spinal procedure or surgery. In addition, in the embodiment illustrated in FIG. 12E, the ligamentum flavum 46 is thickened/enlarged, resulting in spinal stenosis. In particular, the anterior portions of the enlarged ligamentum flavum 46 extend into spinal canal 36, potentially exerting compressive forces on the spinal cord (not shown) that resides within spinal canal 36.

As previously discussed, to relieve compressive forces on the spinal cord and hence relieve the associated symptoms of spinal stenosis, portions of the ligamentum flavum 46 may be separated. However, to percutaneously separate portions of the ligamentum flavum 46 via minimally invasive techniques, the innate structure of vertebral column 10 and each vertebra 12 may present significant imaging challenges. For instance, lateral imaging windows/views of the ligamentum flavum 46 substantially in the coronal plane 56 may be obscured by the various processes of the vertebrae (e.g., transverse processes, superior articular processes, inferior articular processes), and the laminae of each vertebra, etc. Further, some anterior-posterior (A-P) imaging windows/views of the ligamentum flavum 46 substantially in the sagittal plane 54 may also be obscured by the laminae 20. In particular, in the A-P radiographic imaging planes substantially in the sagittal plane 54, the posterior edges of parallel laminae 20 overlap and obscure the ligamentum flavum 46 and the interlaminar space 38, particularly the anterior portions of the ligamentum flavum 46 and the interlaminar space 38 closest to spinal canal 36. However, with an imaging window/view in a plane substantially parallel to the sagittal plane 54, at an angle generally in the direction of arrow 83 shown in FIG. 12B, and slightly lateral to the spinous process 22, interlaminar space 38 and ligamentum flavum 46 may be viewed without significant obstruction from neighboring laminae 20. In other words, imaging windows/views generally aligned with arrow 83 (FIG. 12B) allow for a more direct view of the interlaminar space 38 and the ligamentum flavum 46 from the posterior back surface with minimal obstruction by the vertebrae, or from the laminae.

Typically, the long axes of the substantially parallel laminae (e.g., laminae 20a, 20b, 20c) and interlaminar spaces (e.g., interlaminar spaces 38) are generally oriented between about 60 and about 75 degrees relative to posterior back surface 70. Thus, preferably the imaging means (e.g., x-ray beam, fluoroscopy tube, etc.) is positioned generally in the direction represented by arrow 83, where θ is substantially between about 60 and about 75 degrees relative to the anterior back surface 70. In other words, the imaging apparatus is positioned substantially parallel to the surface of the laminae. The resulting imaging window/view, termed “caudal-cranial posterior view” hereinafter, permits a clearer, more direct, less obstructed view of the interlaminar space 38 and the ligamentum flavum 46 from the general posterior back surface 70. The caudal-cranial posterior view permits a relatively clear view of the interlaminar space 38 and the ligamentum flavum 46 in directions generally along the axial and coronal planes. However, the caudal-cranial posterior view by itself may not provide a clear imaging window/view of the interlaminar space 38 and the ligamentum flavum 46 in directions generally along the sagittal plane 54. In other words, the caudal-cranial posterior view by itself may not provide a clear imaging window/view that can be used to accurately determine the posterior-anterior depth, measured generally along the sagittal plane, of a device across the ligamentum flavum 46.

In some embodiments, an additional imaging window/view, termed “caudal-cranial posterior-lateral view” hereinafter, is employed to provide a clearer, unobstructed view of interlaminar space 38 and ligamentum flavum 46 in directions generally along the axial 52 and coronal 56 planes. The caudal-cranial posterior-lateral view is generated by orienting an imaging means generally at an angle θ relative to the outer surface of the patient and also angling such imaging means laterally in an oblique orientation, revealing a partial lateral view of the interlaminar space 38 occupied by the ligamentum flavum 46 on the anterior side of the lamina and posterior to the underlying dural sac (not shown) and spinal cord (not shown).

By employing at least one of the caudal-cranial posterior views and the caudal-cranial posterior-lateral views, relatively clear imaging windows/views of the interlaminar space 38 and ligamentum flavum 46 in directions along the sagittal 54, coronal 56, and axial 52 planes may be achieved.

FIGS. 12C-E illustrate vertebral column 10 and an instrument 100. Once unobstructed imaging windows/views of the interlaminar space 38 and the ligamentum flavum 46 are established in the manner described above, instrument 100 is employed to percutaneously access the interlaminar space 38 and the ligamentum flavum 46. Instrument 100 may be any suitable device necessary to perform the ligament decompression procedures described herein including without limitation a tissue separation device, a cannula employed to guide a tissue separation device, or combinations thereof. Tissue separation tools and devices are described in more detail below.

More specifically, using images of the interlaminar space 38 and the ligamentum flavum 46 obtained from the desired direction(s), (e.g., caudal-cranial posterior view and the caudal-cranial posterior-lateral view), instrument 100 can be employed to penetrate the skin and soft tissue in the posterior back surface 70 of the patient. In preferred embodiments, the skin entry point for instrument 100 is between about 5 and about 10 cm inferior (caudal to) the posterior surface of the interlaminar space 38 of interest. For instance, if the portion of the ligamentum flavum 46 between lamina 20a and lamina 20b is the area of interest, then instrument 100 may be inserted into the patient's back about 5 to about 10 cm inferior to posterior surface 70 of the interlaminar space 38.

Referring still to FIGS. 12C-E, instrument 100 can be initially inserted into the posterior tissue and musculature of the patient generally parallel to the longitudinal axis of spinal column 10. In other words, the angle β between the posterior back surface 70 and instrument 100 is between about 0 and about 10 degrees when instrument 100 is initially inserted. Further, instrument 100 is preferably inserted into the posterior tissue and musculature of the patient on the same side (ipsilateral) of the median plane as the area of interest (e.g., the targeted portion of ligamentum flavum 46), as best seen in FIG. 11B. Once instrument 100 is inserted into the posterior tissue and musculature of the patient, instrument 100 then may be oriented about 5 to about 90 degrees relative to the posterior back surface 70 of the patient in order to create a trajectory across the ligamentum flavum 46 in the area of interest (see, e.g., FIGS. 12C-E). Furthermore, once an instrument is inserted into the patients posterior back surface 70, the ends of instrument 100 are free to pivot about the insertion location in posterior back surface 70 in the general direction of the axial 52 and the coronal 56 planes, and may be advanced posteriorly or anteriorly generally in the direction of the sagittal 54 plane.

Once inserted into the posterior tissue and musculature of the patient, instrument can be positioned to provide a trajectory across the interlaminar space 38 in the area of interest, generally towards the anterior 71 surface of the lamina 20 superior to the area of interest. For example, if interlaminar space 38 between lamina 20a and lamina 20b is the area of interest, instrument 100 is positioned to provide a trajectory that will allow a cutting instrument to be inserted across interlaminar space 38 between one lamina 20a and another lamina 20b towards the anterior surface of lamina 20a (superior on cephalad 62 lamina).

By switching between the caudal-cranial posterior view and the caudal-cranial posterior-lateral view, or by viewing both the caudal-cranial posterior view and the caudal-cranial posterior-lateral view at the same time, the instrument can be advanced to the ligamentum flavum 46 in the area of interest with more certainty than has heretofore been present. Once the instrument has reached the ligamentum flavum 46, portions of the ligamentum flavum 46 may be separated with a tissue separation device so as to relieve pressure on the spinal nerves 42. If instrument comprises a tissue separation tool, instrument may be inserted into the ligamentum flavum 46 to separate portions of the ligamentum flavum 46. However, if instrument comprises a cannula, instrument will be positioned adjacent the ligamentum flavum 46 in the region of interest and a tissue separation device 100 may be advanced through instrument toward ligamentum flavum 46 and inserted in ligamentum flavum 46 in the region of interest to retract tissue therefrom. In some embodiments, tissue separation can be performed generally from posterior to anterior across the interlaminar space 38 and then laterally along the anterior portion of the ligamentum flavum 46 if desired. The actual depth of the tip of instrument (or any tissue separation device 100 passing through instrument in the case instrument is a cannula) in the general direction of the sagittal 54 plane may be adjusted with guidance from the caudal-cranial posterior-lateral view and appropriate retraction/advancement of instrument and appropriate adjustment of instrument between about 5 and about 90 degrees relative to the posterior back surface 70.

E. Tissue Separation

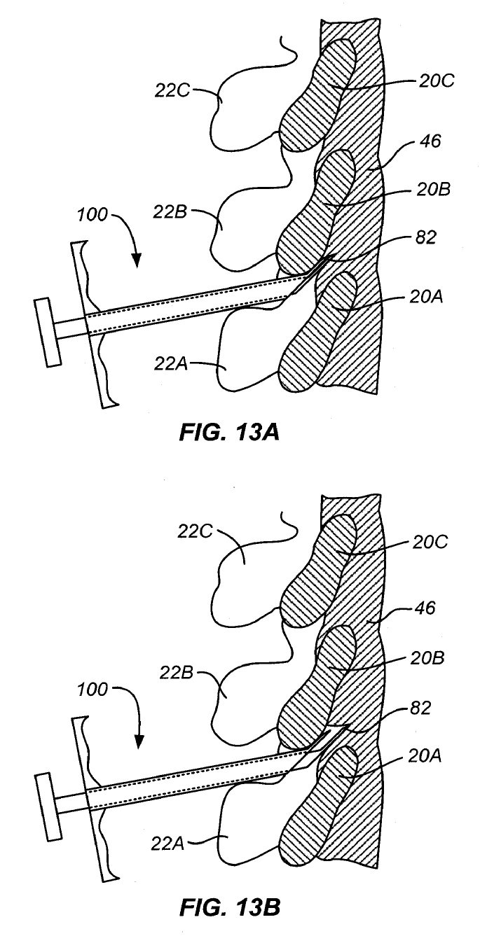

FIGS. 13A-B schematically illustrate the separation of a portion of tissue 82 by device 100. In some embodiments, an instrument, such as a portal or cannula (not shown), may be employed to provide percutaneous access to tissue 82. For instance, tissue separation device 100 may be inserted into and advanced through such a portal or cannula to reach targeted tissue 82. U.S. Publication US 2007/0055263, discloses several tools, devices and methods for employing a portal to provide percutaneous access to a tissue of interest. If a portal or cannula is used to guide device 100, device 100 may be passed through the cannula to reach the tissue of interest.

Regardless of the manner in which tissue separation device 100 reaches the tissue of interest (e.g., by portal or otherwise), device 100 is preferably advanced to the tissue of interest 82 without actuation of the tissue separation device 100. Where the tissue separation device is actuated by an actuator, such as a motor, this would occur with the motor off (i.e., without drive member 132 actuated).

FIGS. 13A-B schematically illustrate the separation of a portion of ligamentum flavum 46 by device 100. In some embodiments, a portal or cannula (not shown) may be employed to provide percutaneous access to ligamentum flavum 46. For instance, ligament separation device 100 may be inserted into and advanced through such a portal or cannula to reach ligamentum flavum 46. U.S. Publication US 2007/0055263 discloses several tools, devices and methods for employing a portal to provide percutaneous access to a tissue of interest. If a portal or cannula is used to guide device 100, device 100 may be passed through the cannula to reach the tissue of interest. As an exemplary embodiment, the method described below will refer to separation of the ligamentum flavum 46 from the lamina of the posterior aspect of the spine. Where the device 100 is passed through a cannula, to reduce the overall profile of the device, it may be desirable to form the upper separation member 112 and lower separation member 122 from a material that has shape memory qualities such that the members 112, 122, can be extended (flattened) as the device is advanced through the cannula, and then assume its tissue separation configuration (shown in FIGS. 6-9) after the distal end of the device 100 exits the distal end of the cannula and is no longer constrained.

Upper and lower separation members 112, 122 are preferably inserted in a closed position as shown in FIGS. 13A-B between ligamentum flavum 46 and interior laminar edge. Device 100 may be inserted such that outer surface of upper member 112 contacts inner surface of lamina 20 while outer surface of lower member 122 contacts ligamentum flavum 46. To separate ligamentum flavum 46 from lamina 20, lower member 122 may be moved in a distal direction to force ligamentum flavum 46 from the lamina 20. In embodiments with inner and outer handles, inner handle 130 may be pulled back in a proximal direction forcing upper member 112 in a proximal direction and apart from lower member 122. Alternatively, outer handle 136 may be pushed in distal 96 direction forcing lower member 122 away from upper member 112 in a distal direction 96 as shown in FIG. 13B. The force of pushing and/or pulling members 112. 122 apart causes separation of the ligamentum flavum 46 from lamina 20. In further embodiments, instead of manually pulling and/or pushing handles as described above, device 100 may use any suitable means to move separation members 112, 122 apart such as a screw or threaded mechanism, ratcheting mechanism, actuator, or combinations thereof. Once the ligamentum flavum 46 and lamina 20 are sufficiently separated, a variety of suitable procedures may be performed on the now exposed surfaces of ligamentum flavum 46 and lamina 20.

Once separation of ligamentum flavum 46 and lamina 20 is no longer desired, upper and lower separation members 112, 122 may be drawn back together in a closed position and retracted from the body of the mammalian patient. Device 100 may be re-inserted and the separation techniques described above may be repeated to separate tissue, ligaments and/or bone in different parts of the mammalian body. Accordingly, although device 100 has been described with respect to the spine, device 100 may be used in other musculoskeletal parts of the body such as without limitation, knee, shoulder, ankle, etc.

The components of tissue separation device 100 (e.g., hollow body 110, inner body 120, separation members 112, 122, etc.) may comprise any suitable material(s) including without limitation metals (e.g., stainless steel, titanium, etc.), non-metals (e.g., polymer, composites, etc.) or combinations thereof. The components of tissue separation device 100 are preferably manufactured from a durable biocompatible material such as titanium or stainless steel, but may alternatively be polymeric.

In addition, the components of tissue separation device 100 may be manufactured by any suitable methods. Examples of suitable methods include casting or molding, machining, laser cutting, EMD, or combinations thereof. In some embodiments, distal tip may be electro polished to for sharpening. The components of tissue separation device 100 may be assembled by any suitable method including without limitation welding, press fitting, or combinations thereof.

Once a user or surgeon has reached the desired hard or bony tissue, device 100 may be switched on. Once motor is actuated, drive pivot member 132 rotates tissue separation member 102, thereby allowing the exposed portion of tissue separation member 102 to begin separating tissue at the target site. Distal end 96 of device 100 may be moved in a proximal and distal (e.g., back and forth) direction to facilitate separation of the tissue. Device 100 may have a switch to alter the direction at which tissue separation member 102 moves. In other words, motor may be switched to move or drive the tissue facing surface of tissue separation member 102 in a distal direction or a proximal direction.

In general, tissue 82 may be any type of tissue to be separated from another tissue within a mammalian patient including without limitation, soft tissue, fat, muscle, or bone. When used to treat spinal stenosis caused by a thickened ligamentum flavum 46, distal end 96 of device 100 is preferably inserted into the stenotic ligamentum flavum 46, preferably posterior to a safety zone 80, in order to safely cut and remove portions of the thickened ligamentum flavum 46 (see FIG. 13), thereby reducing the stenosis.

Device 100 may also be used in conjunction with one or more other devices. The additional devices (not shown) may be used, for example, to first excise soft tissue such as ligaments or muscle and create a passageway to bone tissue. Device 100 may then be inserted into the passageway with the device 100 turned off (e.g., tissue separation member 102 not actuated).

The process of inserting device 100 into tissue, separating portions of tissue, and reinserting device 100 into the body may be repeated until the desired amount of tissue 82 has been separated or a sufficient space has been created. Referring briefly to FIG. 14A, when device 100 is employed to separate portions of thickened ligamentum flavum 46, this process may be repeated until the spinal canal is adequately decompressed. Further, when device 100 is employed to separate portions of thickened ligamentum flavum 46, distal end 96 of device 100 is preferably controlled to remain within ligamentum flavum 46 and not penetrate safety zone 80. Nonetheless, safety zone 80 is preferably provided so that even an inadvertent penetration into epidural space 44 by device 100 will not result in damage to the dural sac 48 or nerves 42.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for separating a first tissue from a second tissue at a surgical site, comprising:
a hollow body having a distal end and a proximal end, wherein the distal end further comprises an upper separation member extendable laterally from the hollow body at a first angle;
an inner member slidably having a distal end and a proximal end and disposed within the hollow body, wherein the distal end of the inner member has a lower separation member extendable laterally from inner member at a second angle, wherein the upper separation member and the lower separation member have a first configuration and a second configuration.

2. The device of claim 1 wherein the first angle is greater than 90 degrees.

3. The device of claim 2 wherein the second angle is the same as the first angle.

4. The device of claim 1 wherein the hollow body and the inner member are substantially tubular.

5. The device of claim 1 wherein the upper separation member further comprises an outer face and an inner face and the lower separation member further comprises an outer face and an inner face and further wherein the separation members are constructed and arranged so that the inner face of the lower separation member has a distance between 3 and 16 mm from the inner face of the outer separation member in the first configuration.

6. The device of claim 1 further comprising a handle attachable to the proximal end of the hollow body.

7. The device of claim 1 wherein the upper separation member and the lower separation member have distal ends which are beveled.

8. The device of claim 1 wherein the upper separation member and the lower separation member are angled to form a sharpened tip.

9. The device of claim 1 further comprising a means for moving the upper separation member and the lower separation member between the open configuration and the closed configuration, the means being located at the proximal end of the hollow body and the inner member.

10. The device of claim 6 further comprising an actuator coupled to a second pivot member wherein the second pivot member is in communication with the first pivot member.

11. The device of claim 6 wherein the actuator is a motor.

12. The device of claim 8 wherein the motor is disposed within the handle.

13. The device of claim 1 wherein the first tissue and the second tissue are connective tissue.

14. The device of claim 13 wherein the first tissue is ligament and the second tissue is bone.

15. The device of claim 14 wherein the first tissue is a laminae of a vertebral body and the second tissue is a ligamentum flavum.

16. A method for treating stenosis in a spine of a patient having a median plane comprising the steps of:
compressing a dural sac in the region of interest by injecting a fluid to form a safety zone and establish a working zone in the region of interest, the safety zone lying between the working zone and the dural sac;
percutaneously accessing an epidural space in the region of interest on a first lateral side of a median plane; and
inserting a device for separating a first tissue from a second tissue at a surgical site, comprising, a hollow body having a distal end and a proximal end, wherein the distal end further comprises an upper separation member extendable laterally from hollow body at a first angle; an inner member having a distal end and a proximal end and slidably disposed within the hollow body, wherein the distal end of the inner member has a lower separation member extendable laterally from inner member at a second angle, wherein the upper separation member and the lower separation member have a first configuration and a second configuration into a tissue in the working zone on the first lateral side of the median plane.

17. The method of claim 16 further comprising the step of generating at least one view of a portion of a spinal canal in a region of interest.

18. The method of claim 17 further comprising using the at least one view to position the tissue excision device during at least part of the step of inserting.

19. The method of claim 16 wherein a portion of a patient's ligamentum flavum occupies the working zone in the region of interest.

20. The method of claim 16 further comprising the step of using the tissue separation device to percutaneously reduce a stenosis on the first lateral side of the median plane.

21. The method of claim 20 further comprising using the at least one view to position the tissue excision device during at least part of the step of using the tissue excision device.

22. The method step of claim 21 further comprising the step of removing at least a portion of the ligamentum flavum in the region of interest.

23. The method of claim 16 further comprising the step of using a tissue excision device to percutaneously reduce a stenosis on a second lateral side of the median plane different than the first lateral side.

24. The method of claim 17 further comprising using the at least one view to position the tissue excision device during at least part of the step of using the tissue excision device.

25. A kit for tissue separation comprising:
a device for separating a first tissue from a second tissue at a surgical site, comprising, a hollow body having a distal end and a proximal end, wherein the distal end further comprises an upper separation member extendable laterally from hollow body at a first angle; an inner member slidably disposed within the hollow body, wherein the distal end of the inner member has a lower separation member extendable laterally from inner member at a second angle, wherein the upper separation member and the lower separation member have a first configuration and a second configuration into a tissue in the working zone on the first lateral side of the median plane; and
packaging.

26. The kit of claim 25 further comprising an injectable medium.

27. The kit of claim 26 wherein the injectable medium is a contrast medium.

28. The kit of claim 27 wherein the contrast medium is a hydrophillic-lipophillic block copolymer gel.

29. The kit of claim 25 further comprising a guide adaptable for use with the device.
